(19) United States
(12) Patent Application Publication
(10) Pub. No.: US 2013/0090663 A1
(43) Pub. Date: Apr. 11, 2013

(54) APPARATUS AND METHOD FOR SIZING A CIRCUMFERENCE

(71) Applicant: James Marvel, Mineola, TX (US)

(72) Inventor: James Marvel, Mineola, TX (US)

(21) Appl. No.: 13/253,671

(22) Filed: Oct. 5, 2011

(51) Int. Cl. A61B 17/56

(52) U.S. Cl. 606/102

(57) ABSTRACT

An apparatus (10) having a hoop (20) housed within a sheath (14) operable to be extended therefrom using a plunger (22) for placement about a joint (12) to obtain a measurement associated with the joint (12). The hoop (20) is of a resilient material and configured for insertion into a patient using a minimally-invasive surgical technique so as to obtain a precise measurement of the joint (12) while minimizing trauma to the patient.

### BACKGROUND

1. Field

Embodiments of the present invention relate to apparatus and methods of sizing a body part in a surgical environment. More particularly, embodiments of the present invention present a hoop that is, upon extension from a sheath and arthroscopic insertion into a body, extendable about an outer or inner perimeter of a bone to yield a measurement of the bone for sizing a subsequently-installed implant.

2. Related Art

The human body contains a large number of joints with varying complexity. The hip and knee joints are two of the most complex joints in the body. Because these joints are weight bearing, they are highly susceptible to damage. The ends of bones associated with the hip and knee joints, as well as many other joints, are coated with articular cartilage, which is smooth and hard, so as to provide the associated bones with a slick surface during normal movement. The articular cartilage has a very low coefficient of friction and can also receive large compressive loads, which makes it vital to ensure ease of movement of the joints and prevent bone on bone contact therebetween. Normal articular cartilage is about 50 times slicker than ice.

Over time, the articular cartilage on bones wears and degenerates, such that it thins or in some joints is completely lost. This is particularly problematic for weight-bearing joints such as the hip and knee. Upon wear of the articular cartilage, the slick, low friction surfaces from the cartilage are lost, and the ends of bones are exposed. Without any protecting articular cartilage, the bones contact each other. This bone on bone contact is painful and is often the end result of osteoarthritis. Additionally, bones can also become hard and sclerotic over time with associated loss of articular cartilage, which further increases the pain.

Many methods have been developed to either replace worn cartilage or otherwise minimize the pain associated with the loss of the articular cartilage. The methods have varying degrees of success but are often accompanied by very extensive and invasive surgery. All invasive methods are costly, often requiring implanting nonbiologic parts within the knee, or, in some instances, human cadaver parts. These methods of treatment also require lengthy rehabilitation, which often leaves the patient in considerable pain.

One method of treatment that has been used is implantation of cadaver menisci. This method has had only limited success and multiple failures. A second method is chondroplasty, or removal of and thinning out the existing damaged cartilage. This method is used to smooth the cartilage to reduce the friction between the bones and remove the flaps of cartilage that have delaminated from the bone. The success of this procedure is limited by the amount of cartilage remaining, and doctors guard against removal of too much of the articular cartilage so as to prevent exposure of the subchondral bone. For older patients or patients with traumatic arthritis of their knees, chondroplasty has only limited application because of the lack of healthy articular cartilage.

If the articular cartilage loss is small, an osteochondral autograft transplant (known as an OATS procedure) can be performed. The OATS procedure requires removing a dowel shaped portion of bone and replacing it with a commensurate dowel shaped portion of articular cartilage from another area of the joint or even a cadaver. The OATS procedure is relatively invasive, has a fairly lengthy rehabilitation time, and has only limited success.

An even further alternative to repairing articular cartilage damage is growing the patient's own cartilage in tissue cultures and placing the newly grown cartilage in the areas of cartilage loss. This is an expensive and often unsuccessful method of treatment.

In the most extreme cases of arthritis, the joint may be artificially resurfaced or even replaced. In artificial joint replacement, the ends of the bones are capped with plastic or metal pieces that are cemented to the ends of the bone. Alternatively, the ends of the bones can be replaced with a biologic ingrowth coating of the metal used, which removes the need for the cement. This procedure is presently the standard approach to treating severe osteoarthritis of the knee.

In most of these procedures, it is desirable to measure one or more of the bones associated with the joint to, for instance, correctly size an implant for use therewith. The standard approach to obtaining such a measurement is utilizing calipers to measure a diameter of the bone. Given that bones are generally not perfectly round, it is necessary to make a number of measurements about a surface of the bone using the calipers and, thereafter, reduce such measurements to a single estimated measurement of the bone. This process of measuring the bone is imprecise, which may result in installation of an implant that is improperly sized with respect to the bone. If the implant is too small, the implant may, for instance, be incapable of absorbing weight transferred from bones associated with the joint and the lifespan of the implant may be shortened by premature failure of the implant. If the implant is too large, the implant may, for instance, interfere with soft tissue adjacent to the implant, which subjects the patient to undue pain. Additionally, this process of measuring the bone is quite invasive and requires an excessive amount of time, which increases trauma to the patient and extends rehabilitation time.

Accordingly, there is a need for a relatively more precise, more efficient, and less invasive apparatus and method for measuring a human bone. The present invention solves the above-described problems and provides a distinct advantage in the art of medical treatments associated with joints. More particularly the present invention provides a new apparatus and method to precisely measure a human bone, body part, footprint, or other area in a manner that is minimally invasive with minimal trauma so that an implant may be appropriately sized prior to deployment of the implant within the patient.

### SUMMARY

The following brief summary is provided to indicate the nature of the subject matter disclosed herein. While certain aspects of the present invention are described below, the summary is not intended to limit the scope of the present invention. Embodiments of the present invention provide an irrigation system and method that does not suffer from the problems and limitations of conventional irrigation systems such as those set forth above.

Embodiments of the present invention provide an apparatus having a sheath, a hoop housed in a sheath, and a plunger coupled with the sheath and operable to extend the hoop about a joint to obtain a measurement associated with the joint.

In more detail, embodiments of the present invention provide a housing structure that defines a chamber, a selectively configurable hoop housed within the chamber and operable to be extended from the chamber, and a controller connected to the hoop, wherein the controller is operable to cause the hoop to be extended from and retracted into the chamber. A position of the controller relative to the housing structure indicates a measurement of at least a portion of a perimeter of the bone.

The hoop may be selectively configurable between a stored configuration in which a majority or an entirety of the hoop is retracted into the chamber and a use configuration in which the hoop is at least partially extended out of the chamber. The hoop may have a configuration defined by a degree of extension of the hoop from the chamber. The hoop may have a circumference defined by the position of the controller relative to the housing structure. A size of the hoop may be determined by how far the hoop is extended from the chamber. The hoop may be of a resilient material such that, when the hoop is at least partially extended out of the chamber, an extended portion of the hoop is generally circular in shape.

The controller may include an elongated rod extending from a first end of the housing structure. The rod may be operable to extend and retract the hoop out of and into a second end of the housing structure. The rod may include indicia thereon operable to indicate the measurement based on the position of the controller relative to the housing structure.

The apparatus may further include a closure on the first end of the housing structure to form a seal therebetween. The closure may include a slit therethrough that is sized and shaped to slidably receive a portion of the rod to permit connection of the rod to the hoop within the chamber of the housing structure.

Embodiments of the present invention also provide a method of measuring a bone including the steps of providing an apparatus having a housing structure, a chamber within the housing structure, and a hoop housed within the chamber in a first configuration; positioning the apparatus adjacent to a bone; manipulating a controller secured to the housing structure to cause the hoop to be at least partially extended from the chamber and toward the bone so that the hoop assumes a second configuration; and observing indicia on the apparatus based on a position of the controller relative to the housing structure to obtain a measurement of the bone.

The first configuration of the hoop may be a compressed configuration in which a majority or an entirety of the hoop is retracted into the chamber and the second configuration of the hoop may be a use configuration in which at least a portion of the hoop is extended out of the chamber and the portion of the hoop has a decompressed configuration. The decompressed configuration of the portion of the hoop may have a generally circular shape.

The method may further include the step of calibrating the indicia based on whether an interior perimeter or an exterior perimeter of the hoop is to be used to measure the bone. The second configuration of the hoop may include a relationship to the bone that is one of (i) about an outer circumference of the bone, (ii) about an inner circumference of the bone, and (iii) substantially aligned with a footprint of the bone. The second configuration of the hoop may have a variable metric that is defined by a degree of extension of the hoop from the chamber. The variable metric may be a circumference or diameter of the hoop. The controller may include an elongated rod extending from a first end of the housing structure. The rod may be operable to extend and retract the hoop out of and into a second end of the housing structure. The housing structure may include a closure at a first end of the housing with a slit through the first end of the housing. The slit may be sized and shaped to slidably receive a portion of the rod to permit connection of the rod to the hoop within the chamber of the housing structure.

Additional aspects, advantages, and utilities of the present invention will be set forth in part in the description which follows and, in part, will be obvious from the description, or may be learned by practice of the present invention.

### BRIEF DESCRIPTION OF THE DRAWINGS

Embodiments of the present invention are described in detail below with reference to the attached drawing figures, wherein:

FIG. 1 is an elevated side view of an apparatus having a hoop extended from a sheath and engaged with a detracted or dislocated femur head of a hip joint, in accordance with an exemplary embodiment of the present invention, with the apparatus in phantom in a disengaged orientation;

FIG. 2 is a top left rear side perspective view of the apparatus illustrated in FIG. 1 with the hoop substantially extended;

FIG. 3 is a top left front side perspective view of the apparatus illustrated in FIG. 1 with the hoop substantially retracted;

FIG. 4 is a bottom right front side perspective view of the apparatus illustrated in FIG. 1 with the hoop substantially retracted;

FIG. 5 is a top left front side perspective view of the apparatus illustrated in FIG. 1 with the hoop extended about the joint;

FIG. 6 is a bottom right front side perspective view of the apparatus illustrated in FIG. 1 with the hoop partially extended;

FIG. 7 is a top plan view of the apparatus illustrated in FIG. 1 with the sheath in cross-section and the hoop substantially retracted therein;

FIG. 8 is a top plan view of the apparatus illustrated in FIG. 1 with the sheath in cross-section and the hoop partially extended therefrom; and

FIG. 9 is a top left rear side perspective view of the apparatus illustrated in FIG. 1 with a sleeve exploded from the sheath.

The drawing figures do not limit the present invention to the specific embodiments disclosed and described herein. The drawings are not necessarily to scale, emphasis instead being placed upon clearly illustrating the principles of the present invention.

### DETAILED DESCRIPTION

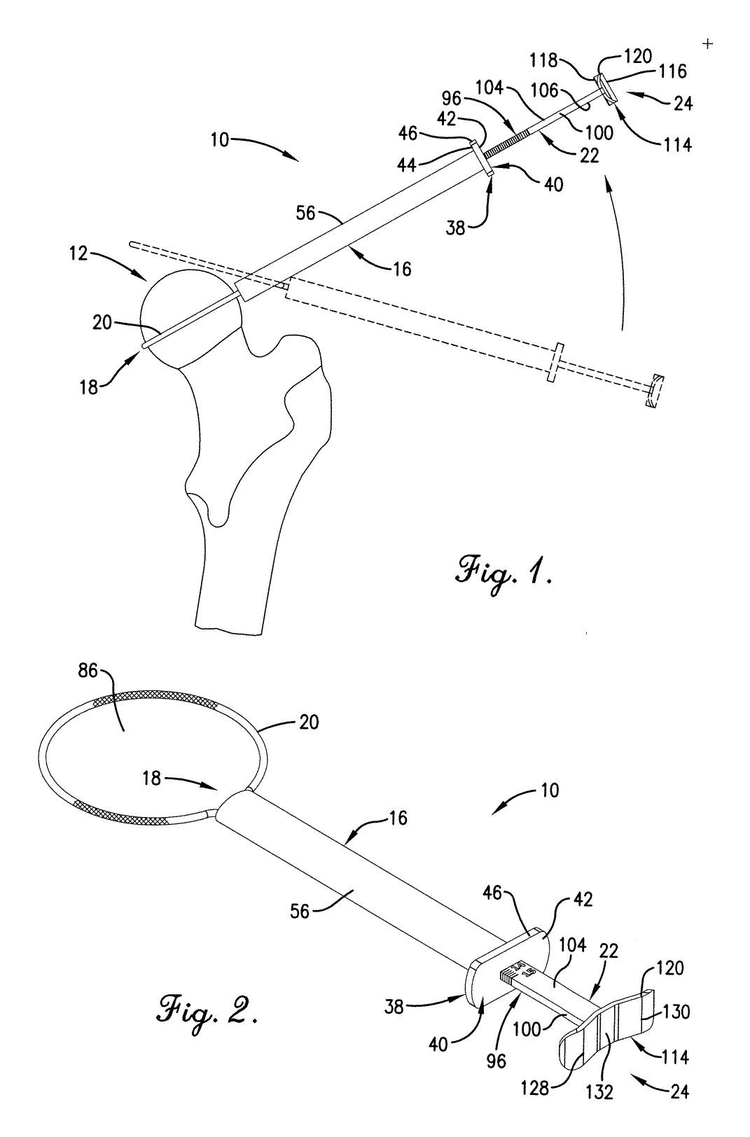

Turning now to the drawing figures, and particularly FIG. 1, a sizing apparatus 10 for a bone constructed in accordance with embodiments of the present invention is illustrated. The apparatus 10 is configured to be utilized with any bone, e.g., bones associated with a knee or hip joint. The apparatus 10 is operable to measure an exterior surface of the bone, e.g., an outer perimeter or circumference of the bone, an interior surface of the bone, e.g., an inner perimeter, concavity, or socket of the bone, or an area related to the bone, e.g., a footprint of the bone or space between bones. In this manner, the apparatus 10 provides precise sizing for an implant to be subsequently installed on or otherwise utilized with the bone. Embodiments of the present invention will be discussed in use with respect to a detracted or dislocated femur head of a hip joint 12, although it is to be understood that the present invention is equally useable with other joints.

The apparatus 10 of the exemplary embodiment of the present invention broadly includes a sheath 14 within a sleeve 16 that cooperatively defines a patient or distal end 18 of the apparatus 10, and a band or hoop 20 that is selectively extendable from the sheath 14 via a telescoping controller or plunger 22. The plunger 22 extends from a surgeon or proximal end 24 of the apparatus 10 to permit manipulation of such by a user or surgeon. In this manner, the surgeon may introduce the hoop 18 into a patient via arthroscopic insertion and maneuver the hoop 18 to measure the actual joint 12 and/or an area related to the joint 12. A size of the hoop 18, particularly a circumference or diameter of the hoop 18, may be varied by expanding and retracting the hoop 18 via the plunger 22 to perform the measurement. For instance, the hoop 18 may be cinched tightly around the joint 12 via the plunger 22 to yield an outer circumferential measurement of the joint 12.

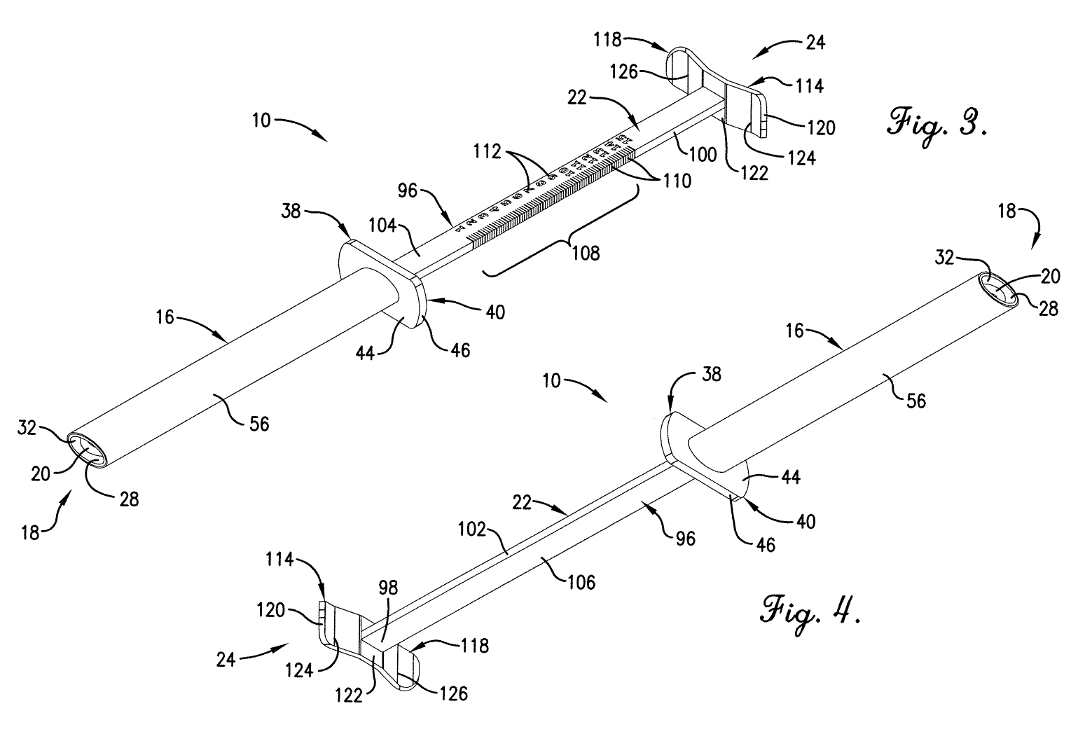

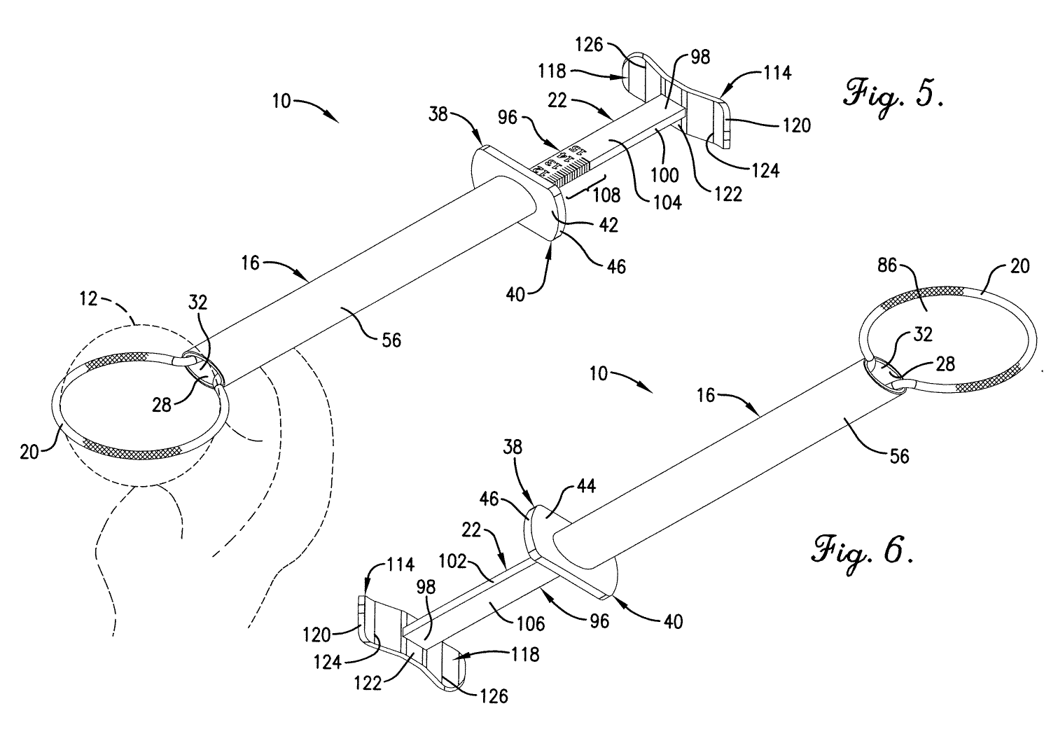

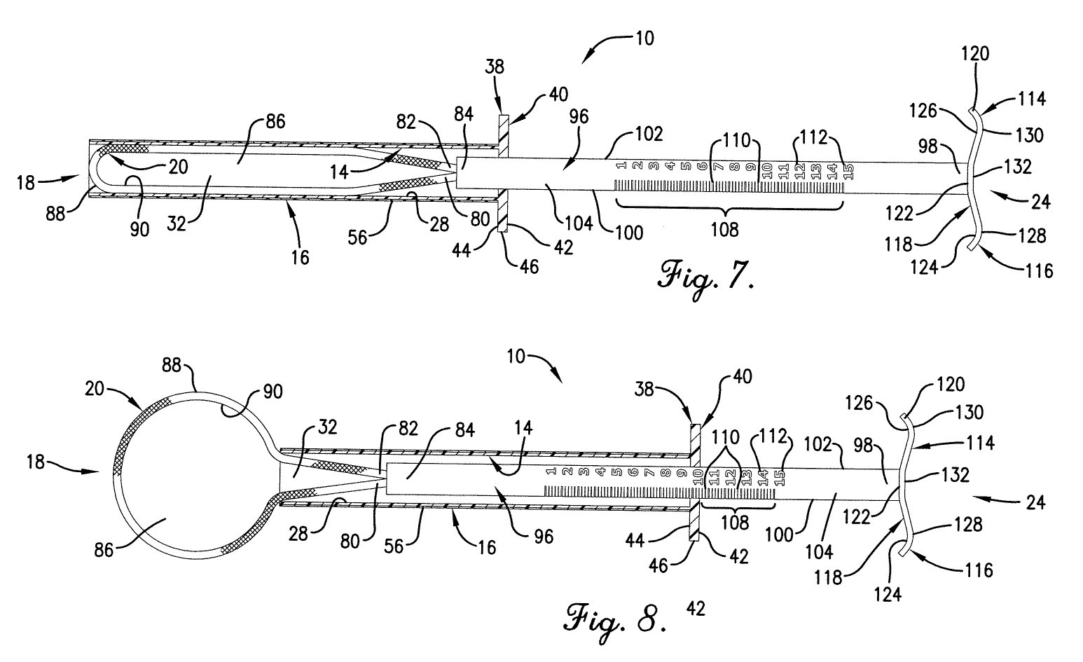

Turning to FIGS. 2-8, the sheath 14 includes an elongated cylindrical sheath wall 26 having interior and exterior surfaces 28, 30. The sheath wall 26 defines a sheath chamber 32 that is sized and shaped to house a majority and preferably an entirety of the hoop 20. It is foreseen, however, that the chamber 32 may house any portion of the hoop 20 without deviating from the scope of the present invention.

The chamber 32 has an open end 34 at an end of the sheath 14 adjacent to the patient end 18 of the apparatus 10, and a closed end 36 at an end of the sheath 14 adjacent to the surgeon end 24. In the exemplary embodiment, the sheath 14 is made of stainless steel. It is foreseen, however, that the sheath 14 may be made of any like material that is durable and easily sterilized without deviating from the scope of the present invention.

The closed end 36 of the sheath 14 is closed by a cap or closure 38 to form a seal therebetween. The closure 38 includes a top wall 40 with upper and lower surfaces 42, 44, and a circumferential edge 46 thereabout. Extending through the upper and lower surfaces 42, 44 is a slit 48 that will be discussed hereafter. In the exemplary embodiment, the closure 38 is formed integrally with the sheath 14 using the same material as the sheath 14, that is, stainless steel. It is foreseen, however, that the closure 38 may be selectively removable from the sheath 14 to, for example, permit access to the sheath chamber 32 without deviating from the scope of the present invention. It is further foreseen, however, that the sheath 14 may be made of any like material that is durable and easily sterilized without deviating from the scope of the present invention.

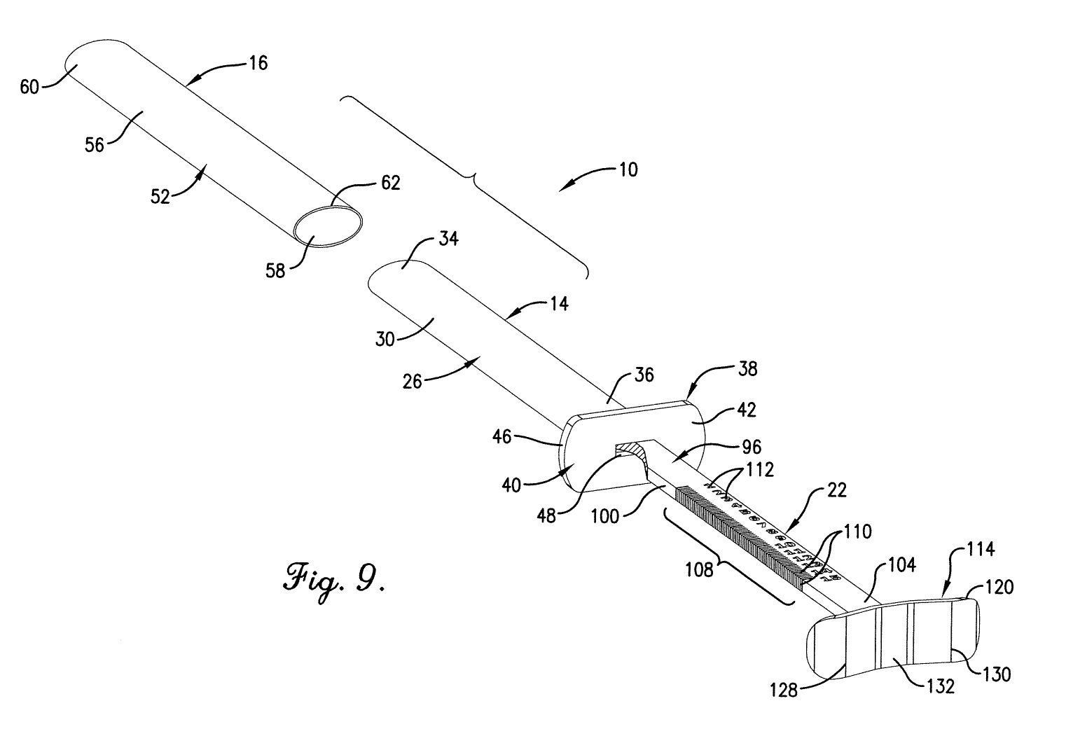

The sleeve 16 includes an elongated cylindrical sleeve wall 52 having interior and exterior surfaces 54, 56, as illustrated by FIG. 9. The sleeve wall 52 defines a sleeve chamber 58 that is sized and shaped to slidably receive the sheath 14 therein. The sleeve chamber 58 has a first open end 60 at an end of the sleeve 16 adjacent to the patient end 18 of the apparatus 10, and a second open end 62 at an end of the sleeve 16 adjacent to the surgeon end 24. When fitting the sleeve 16 over the sheath 14, the interior surface 54 of the sleeve 16 is configured to slide along the exterior surface 30 of the sheath 14 until the cylindrical sleeve wall 52 abuts the lower surface 44 of the closure 38. In this manner, the sleeve 16 is both removably and slidably secured to the sheath 14.

The sheath 14 and sleeve 16 are similarly sized and shaped and, in the exemplary embodiment, are generally oval in shape across a width of the sheath 14 and sleeve 16 and are preferably approximately six inches long and a half inch wide. It is foreseen, however, that sheath 14 and sleeve 16 may be sized and/or shaped differently, such as but not limited to circular or rectangular, without deviating from the scope of the present invention.

The hoop 20 is a cable having first and second connecting ends 80, 82. Each of the ends 80, 82 are secured together at a common end 84 of the plunger 22 via welding or the like. In this manner, the hoop 20 forms an enclosed area 86 and includes an outermost perimeter 88 and an innermost perimeter 90, as best illustrated in top-plan view FIG. 8. The hoop 20 is manufactured from woven stainless steel or the like having two millimeter diameter and a degree of resiliency that causes the hoop 20 to assume a configuration depending on a position of the hoop 20 in relation to the sheath chamber 32. In this manner, the hoop 20 is selectively configurable between a first stored configuration and a second use configuration using the plunger 22.

In the stored configuration, the hoop 20 is completely retracted into the sheath chamber 32 with the interior surface 28 of the sheath chamber 32 abutting the outer perimeter 88 of the hoop 20 so that the hoop 20 is compressed by the sheath 14 into a generally oval shape. In the use configuration, the hoop 20 is at least partially extended from the sheath chamber 32. The resiliency of the hoop 20 causes an extended portion of the hoop 20 to decompress and assume a generally circular shape while a retracted portion of the hoop 20 remains compressed by the sheath chamber 32, as respectively illustrated in FIGS. 7 and 8.

The extended portion of the hoop 20, and particularly the enclosed area 86 thereof, has a variable circumference that may be increased and decreased by respectively extending or retracting the hoop 20 with respect to the sheath chamber 32 to accommodate a wide variety of variously-sized joints. It is foreseen that the hoop 20 may be replaced, in its entirety, with longer or shorter hoops, so that apparatus 10 may be used with body parts. For instance, the hoop 20 is of an ideal length for use with the hip joint 12 while a relatively shorter hoop may be better suited for use with a knee joint.

The length of the sheath 14 is determined based on the length of the hoop 20, to enable the sheath chamber 32 to house the hoop 20 in its entirety when fully retracted therein by the plunger 22. The hoop 20 to sheath 14 ratio is preferably 1:2, and more preferably approximately 1:1.75.

The connecting ends 80, 82 of the hoop 20 are secured to the end 84 of the plunger 22, and particularly to an elongated rod 96 of the plunger 22. The rod 96 extends through the slit 48 in the closure 38 and is joined with the ends 80, 82 of the hoop 20 within the sheath chamber 32. The rod 96 extends longitudinally from the sheath chamber 32, through the slit 48, and to another end 98 of the plunger 22. The rod 96 includes opposing side surfaces 100, 102 and opposing top and bottom surfaces 104, 106. In the exemplary embodiment, the side surface 100 and the top surface 104 has measurement indicia 108, which includes one or more hatch marks 110 and one or more corresponding numerals 112 that relate to centimeters and/or millimeters. It is foreseen, however, that any one or more of the surfaces 100, 102, 104, 106 may include measurement indicia 108 having marks 110 or numerals 112, or both without deviating from the scope of the present invention.

The slit 48 and rod 96 are sized and shaped to tightly yet slidably mate with each other so as to not compromise the integrity of the seal between the sheath 14 and the closure 38. The rod 96 is slidable with respect to the slit 48 along an entire length of the rod 96 between a maximum degree of extension, in which the rod 96 is almost entirely extended from the sheath 14 and substantially exposed from the sheath 14, and a minimum degree of extension, in which the rod 96 is entirely extended into the sheath 14 and substantially concealed within the sheath 14.

The minimum degree of extension is defined by a handle 114 at the end 98 of the plunger 22. The handle 114 defines the surgeon end 24 of the apparatus 10, and includes upper and lower surfaces 116, 118 with a common perimeter 120. The lower surface 118 of the handle 116 includes a connecting surface 122 that connects to the rod 96. The upper and lower surfaces 116, 118 have a common contour. On either side of the connecting surface 122 are lower inward bends 124, 126, that each translate to outward bends 128, 130 on the upper surface 116, respectively. Between the outward bends 128, 130 on the upper surface is a depressed level surface 132 that is opposite to the connecting surface 122.

The handle 114, and particularly the lower surface 126 thereof, is operable to abut the closure 38, and particularly the upper surface 42 thereof, when the rod 96 is entirely extended into the sheath 14 and substantially concealed therein. In this manner, the handle 114 provides a backstop, thereby preventing the rod 96 from extending completely into the sheath 14. The maximum degree of extension is defined by the hoop 20, which has a circumference greater than a width of the slit 48. In this manner, the hoop 20 provides a backstop, thereby preventing the rod 96 and hoop 20 from extending completely out of the sheath 14.

An exemplary use of the apparatus 10 during arthroscopic surgery of joint 12 will now be described. With the plunger 22 fully extended and the hoop 20 fully retracted into the sheath chamber 32, the surgeon aligns the patient end 18 with a small incision made on a skin surface of the patient adjacent to the joint 12. Preferably, the sleeve 16 is secured to a receiver that stabilizes the apparatus 10 with respect to the patient and allows a tip of the sleeve 16 to extend slightly through the incision into the patient. The apparatus 10 may be disconnected from the sleeve 16 to facilitate connection of the sleeve 16 with the receiver and subsequently reconnected therewith post connection.

Upon stabilization of the apparatus 10, the surgeon manipulates the plunger 22 using the handle 114 to cause the hoop 20 to be arthroscopically inserted into the patient and in closer proximity to the joint 12. During insertion, the hoop 20 remains compressed by the sheath chamber 32 until the hoop 20 passes through the incision and out of the sheath chamber 32, at which point, the resilient material of the hoop 20 allows it to decompress into a generally circular shape. It is foreseen that friction reducing lubricant or fluid may be utilized to facilitate insertion of the hoop 20 via a fluid line attached to the apparatus 10. Alternatively, the fluid may be inserted into the patient prior to use of the apparatus 10. The seal between the sheath 14 and closure 38 is operable to prevent escape of any such fluid as well as any bodily fluid of the patient.

The contours of the handle 114, and particularly the bends 124, 126 and surface 132 of the handle 114, facilitate secure grasping of the handle 114 by the surgeon with one hand while the other hand grasps the sleeve 16 and the closure 38. To observe movement of the hoop 20, the surgeon may utilize a small fiberoptic camera or arthroscope inserted into another small incision in the patient.

In the exemplary embodiment, the hoop 20 is maneuvered to fit around an outer circumference of joint 12. While maneuvering the hoop 20, it may be necessary to further extend or retract the hoop 20 using the plunger 22 to adjust the size of the enclosed area 86 in view of the outer circumference of the joint 12. Additionally, it may be necessary to pivot the apparatus 10, as illustrated in FIG. 1, to lasso the joint 12. Once the joint 12 is within the enclosed area 86 and the hoop 20 surrounds the joint 12, the hoop 20 is cinched around the joint 12 by extending the plunger 22 away from the sheath 14. The surgeon, after ensuring that the hoop 20 is sufficiently cinched around the joint 12 via the arthroscope, may then observe the indicia 108 on the rod 96 to learn the precise measurement of the joint 12. For instance, if the numeral 112 of the indicia 108 most approximate to the closure 38 is fourteen, as illustrated in FIG. 2, then it is known that the circumference or diameter of the joint 12 is fourteen centimeters or millimeters. For more precise measurements, the marks 110 may be used in combination with the numerals 112.

The measurement of the joint 12 may then be recorded and the hoop 20 and apparatus 10 removed from the patient so that an appropriately-sized implant may be matched for use with the joint 12 and installed in the patient. The sleeve 16 may be disconnected from the apparatus 10 and remain attached to the receiver for use with subsequent procedures.

The surgeon may perform additional measurements on adjacent portions of the joint 12. For instance, the hoop 20 may be utilized to measure the inner circumference of an acetabulum, that is, a concave surface of a pelvis that receives the joint 12. It is foreseen that other measurements may be used in lieu of or in addition to measurement of an outer circumference. For instance, the measurement of the inner circumference as well as measurement of a footprint of a joint, for instance, in knee surgery. Particularly, U.S. Pat. No. 7,976,578 to Marvel, the disclosure of which is incorporated herein by reference in its entirety, discloses a buffer for placement in a human knee between the femur and tibia. The present invention may be utilized to measure the area between the femur and tibia prior to insertion of the buffer, so that an appropriately-sized buffer may be placed therebetween.

It is foreseen that it may be necessary to calibrate the apparatus 10 depending on the manner in which the hoop 20 is used to measure objects. For instance, measurements of inner and outer circumferences may require a slight adjustment of the indicia 108 to account for the width of the hoop 20. The indicia 108 may include additional marks 110 and/or numerals 112 to account for the manner in which the hoop 20 is used to measure objects. For instance, additional marks 110 and/or numerals 112 that account for the width of the hoop 20 may be placed on any one or more of the used or unused surfaces 100, 102, 104, 106 without deviating from the scope of the present invention.

In this manner, the present invention provides an efficient method to precisely measure a bone, body part, footprint, or other area in a manner that is minimally invasive with minimal trauma so that an implant may be appropriate sized prior to deployment of the implant within the patient. Although the present invention has been described with reference to the preferred embodiment illustrated in the attached drawing figures, it is noted that equivalents may be employed and substitutions made herein without departing from the scope of the present invention as recited in the claims.

Having thus described the preferred embodiment of the present invention, what is claimed as new and desired to be protected by Letters Patent includes the following:

1. An apparatus to measure a perimeter of a bone comprising:
a housing structure that defines a chamber;
a selectively configurable hoop housed within the chamber and operable to be extended from the chamber, said hoop configured to be positioned about the perimeter of the bone;
a controller connected to the hoop, the controller operable to cause the hoop to be extended from and retracted into the chamber; and
indicia operable to indicate a measurement of at least a portion of the perimeter of the bone when the hoop is extended from the chamber.

2. The apparatus of claim 1, wherein the hoop is selectively configurable between a stored configuration in which the hoop is substantially retracted into the chamber and a use configuration in which the hoop is at least partially extended out of the chamber.

3. The apparatus of claim 1, wherein the hoop has a circumference defined by the position of the controller relative to the housing structure.

4. The apparatus of claim 1, wherein the hoop is of a resilient material such that, when the hoop is at least partially extended out of the chamber, an extended portion of the hoop is substantially circular.

5. The apparatus of claim 1, wherein the controller includes an elongated rod extending from a first end of the housing structure, the rod operable to extend and retract the hoop out of and into a second end of the housing structure.

6. The apparatus of claim 5, wherein the indicia indicates the measurement based on the position of the controller relative to the housing structure.

7. The apparatus of claim 5, further comprising:
a closure on the first end of the housing structure, the closure forming a seal on the first end of the housing structure.

8. The apparatus of claim 7, wherein the closure includes a slit therethrough, the slit sized and shaped to slidably receive a portion of the rod to permit connection of the rod to the hoop within the chamber of the housing structure.

9. A method of measuring a perimeter of a bone comprising the steps of:
providing an apparatus with a housing structure having a chamber within the housing and a selectively-configurable hoop at least partially housed within the chamber of the housing structure in a first configuration;
positioning the apparatus adjacent to a bone;
manipulating a controller coupled with the housing structure to cause the hoop to be at least partially extended from the chamber and toward the bone so that the hoop assumes a second configuration; and
observing indicia on the apparatus based on a position of the controller relative to the housing structure to obtain a measurement of at least a portion of the perimeter of the bone.

10. The method of claim 9, wherein the first configuration of the hoop is a compressed configuration in which the hoop is substantially retracted into the chamber and the second configuration of the hoop is a use configuration in which at least a portion of the hoop is extended out of the chamber and the portion of the hoop has a decompressed configuration.

11. The method of claim 10, wherein the decompressed configuration of the portion of the hoop is generally circular.

12. The method of claim 9, wherein the indicia is on the controller to indicate a measurement associated with the second configuration of the hoop.

13. The method of claim 9, wherein the second configuration of the hoop includes a relationship to the bone that is one of (i) about an outer circumference of the bone, (ii) about an inner circumference of the bone, and (iii) substantially aligned with a footprint of the bone.

14. The method of claim 9, wherein the second configuration of the hoop has a variable metric that is defined by a degree of extension of the hoop from the chamber.

15. The method of claim 14, wherein the variable metric is a circumference or diameter of the hoop.

16. The method of claim 9, wherein the controller includes an elongated rod extending from a first end of the housing structure, the rod operable to extend and retract the hoop out of and into a second end of the housing structure.

17. The method of claim 16, wherein the housing structure includes a closure at a first end of the housing with a slit through the first end of the housing, the slit sized and shaped to slidably receive a portion of the rod to permit connection of the rod to the hoop within the chamber of the housing structure.

18. An apparatus to measure a perimeter of a bone, the apparatus comprising:
a housing structure having a chamber;
a selectively-configurable hoop operable to be at least partially housed within the chamber;
a controller extending from an end of the chamber and connected to the hoop, the controller operable to at least partially extend the hoop from another end of the chamber to the perimeter of the bone; and
indicia on the controller to indicate a measurement of the perimeter of the bone while the hoop is at least partially extended from the another end of the chamber.

19. The apparatus of claim 18, wherein the measurement of the perimeter of the bone is an inner circumference of a portion of the perimeter of the bone.

20. The apparatus of claim 18, wherein the measurement of the perimeter of the bone is an outer circumference of a portion of the perimeter of the bone.
